(19) United States
(12) Patent Application Publication
(10) Pub. No.: US 2007/0016307 A1
(43) Pub. Date: Jan. 18, 2007

(54) ABSORBABLE MEDICAL ELEMENT SUITABLE FOR INSERTION INTO THE BODY, IN PARTICULAR AN ABSORBABLE IMPLANT

(76) Inventors: Hanngorg ZIMMERMANN, Gossweinstein (DE); Markus Heinlein, Gossweinstein (DE)

Correspondence Address: MCGLEW & TUTTLE, PC, P.O. BOX 9227, SCARBOROUGH STATION, SCARBOROUGH, NY, 10510-9227, US

(21) Appl. No.: 11/456,331

(22) Filed: Jul. 10, 2006

(30) Foreign Application Priority Data
Jul. 13, 2005 (DE) 10 2005 032 604.8

(51) Int. Cl. A61F 2/02

(52) U.S. Cl. 623/23.75; 606/77; 435/402

(57) ABSTRACT

An absorbable implant comprises a basic compound made of a material which can be absorbed by the body of the implant recipient. A coating which contains titanium partially covers the basic compound, so that the latter comprises coating-free zones for the body to engage and absorb.

### BACKGROUND OF THE INVENTION

1. Field of the Invention

The invention relates to absorbable medical elements suitable for use in the body—in particular an absorbable implant—with a basic compound made of a material which is absorbable in the body of the implant recipient. The possible forms of these medical elements can be used for all appropriate medical applications, such as bolts and plates for bone anchorage, suture material, suture clips, bone replacement granulate, stents, soft tissue reinforcing implants, such as hernia meshes, clips, anchors and staples for anchoring implants, membrane systems as used in dentistry, tubes to replace so-called “nerve tracks”, or base material for use in tissue engineering. For reasons of clarity, these medical elements will be referred to collectively below as “implants”.

2. Background Art

The background to the invention is that absorbable synthetic substances are used in many areas of implant surgery where a temporary stabilisation of any type of tissue such as soft tissue or bone is required, or when an implant is to be used for a limited period. For example, implants are suitable for anchoring a bone fracture when bone splints and bolt systems are used while the bone is self-regenerating. Here, the bone—as is common with a normal fracture—must first be immobilised in order to enable it to grow back together. Then, the anchorage in the form of a foreign substance is no longer necessary, and standard implants made of a durable material must be removed during a post-operation.

An additional intervention of this nature can be avoided when an absorbable material is used for implants such as nails or tracks, which after a certain period of between a few weeks and several months, which can be predetermined by selecting appropriate materials, decompose into the components water and carbon dioxide, which are compatible with the body, whereby the latter are excreted from the body without any problem.

Another example is the use of absorbable suture material, which is suitable for use in nearly all areas of wound medicine. The aim is to close the wound in such a manner that the wound is able to heal with initial adequate tensile strength of the thread. As the body's own tissue stabilises, the tensile strength of the suture material gradually decreases due to the absorption process. After a certain period of time, it finally disintegrates completely.

Finally, the field of tissue engineering should also be mentioned, where porous supporting compounds made of absorbable polymers are created. These are then inoculated with a cell culture such as bone or soft tissue cells. Within the porous structure, which provides a large adsorption surface for the cells, rapid, forming growth of the cells is guaranteed. The initial stability of the structure and thus the shaping is first achieved by the absorbable supporting compound. After a certain growth phase, its own resilience increases and the base synthetic material of the supporting compound in turn gradually disintegrates until only pure cell tissue or bone material is present.

The problem with the absorbable implants according to the prior art is the fact that the implant is identified as being a foreign substance by the recipient body due to its material. This does not usually lead to the rejection reactions which can be observed with organ implants or synthetic implants, but an adsorption and the growth of the body's own cells in the area of the implant can be impaired as a result.

### SUMMARY OF THE INVENTION

Against this background, the object of the invention is to improve an absorbable implant in such a manner that with adequate absorption properties, an adsorption and a corresponding growth of the body's own cells in the area of the implant are supported.

This object is attained by an absorbable medical element suitable for insertion into the body, in particular an implant, comprising a basic compound, which at least partially consists of a material which is absorbable in the body of the implant recipient, a non-absorbable coating of a biocompatible material is applied, which only partially covers this. In this way, the basic compound comprises zones which are free of the coating for a resorption attack of the body of the implant recipient.

Due to the partial coating of the implant according to the invention, an adsorption and the growth of the body's own cells is supported on the one hand, which results in the faster healing of the implant and an optimisation of the cell adsorption. Furthermore, the absorbability can be controlled by the partial coating. On the coated section of the implant, namely, the absorption is decelerated.

Preferred embodiments of the absorbable implant with partial coating are outlined in the subordinate claims and in the description below, where the corresponding features, particularities and advantages are explained in greater detail with reference to the appended drawings.

### BRIEF DESCRIPTION OF THE DRAWINGS

FIG. 1 shows a highly schematic cross-section through an implant with a titanium coating on one side,

FIG. 1B shows an enlarged detail section from FIG. 1A,

FIG. 2A shows a highly schematic cross-section through an implant with a titanium coating on all sides and with interruptions

FIG. 2B shows an enlarged detail section from FIG. 2A,

FIG. 3A shows a highly schematic cross-section through an implant with a thin titanium rudimentary coating, and

FIG. 3B shows an enlarged detail section from FIG. 3A.

### DESCRIPTION OF THE PREFERRED EMBODIMENTS

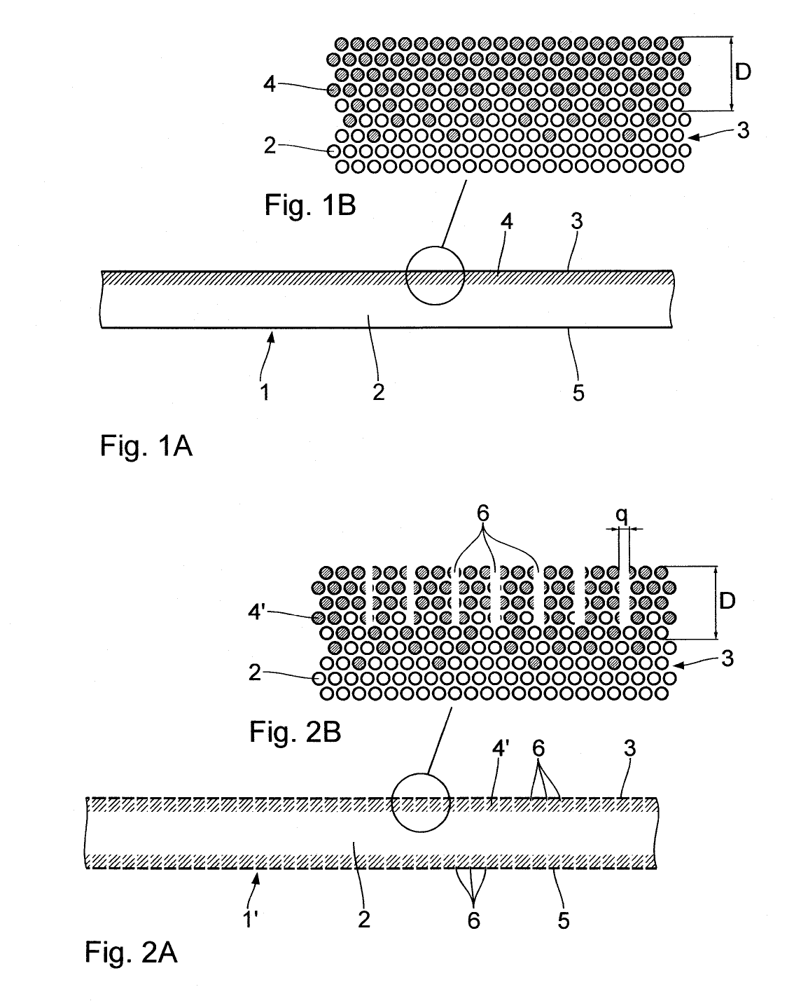

FIGS. 1A and B show an implant 1 such as can be realised by a bone splint, for example. It comprises a basic compound 2, which can consist of a standard absorbable synthetic material such as polylactides, their copolymers, polyglycolides, their copolymers, polydioxanones, proteins such as casei or collagen, tricalcium phosphates and similar substances. It is also possible to produce the basic compound only partially from an absorbable material, such as a hernia mesh, of which half consists of polypropylene (not absorbable) and half of an absorbable material.

The side 3 of the basic compound 2 which faces upwards in FIGS. 1A and B is provided with a continuous coating 4 of a material which contains titanium. This side 3 with the coating 4 e.g. of a bone splint implant 1 lies in an implanted condition in such a manner that it faces the bone which is to be stabilised, so that there, due to the titanium surface which is highly biocompatible, the bone cell growth mentioned above on the bones which are to be stabilised is supported.

On the side 5 of the bone splint which is turned from the other side 3, the bio-absorbable material of the basic compound 2 is completely open and is thus fully exposed to the absorption attack of the body. This accordingly leads to a reliable disintegration of the implant with the specified time constant.

The thickness D of the coating 4 can be dispersed broadly between 5 and 700 nm, preferably between 10 and 100 nm. A practical level is approx. 30 to 50 nm. The coating itself is applied using a PACVD procedure such as that described comprehensively in EP 0 897 997 B1, for example. In this respect, no further explanation of the coating procedure is required here. As an alternative, other suitable coating procedures such as a sol-gel procedure can be used, depending on the coating material.

The embodiment of the implant 1′ shown in FIG. 2 can for example be a stent or a nerve tube. With this implant 1′, a basic compound 2 is in turn provided which is made of an absorbable material listed above, onto which a coating 4′ made of a material which contains titanium is applied on all sides. Due to the interrupted coating 4′ on all sides, the post-operative cell colonisation of the implant 1′ can be supported. In order to form the coating-free zones, the basic compound 2 is marked in those places where later the interruptions 6 in the coating 4′ are to be located. When the coating 4′ is applied—in turn using the above-mentioned PACVD procedure, for example—the interruptions 6 in the coating 4′ are left open and are uncovered when the masking is removed. The open cross-section q of the interruptions can range from several micrometers to a few millimetres in size. The thickness D of the coating 4′ in turn corresponds to approximately 30 to 50 nm.

As not shown in greater detail in the Figures, the interruptions 6 can also be realised subsequently following the application of a continuous coating 4′ by removing the coating 4′ accordingly.

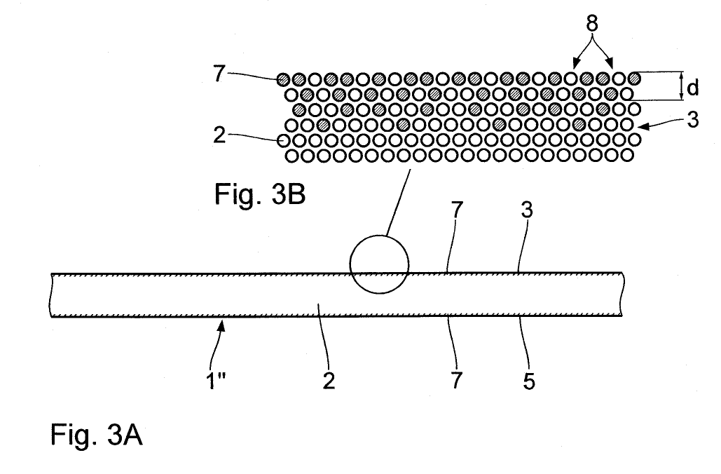

With the implant 1″ shown in FIGS. 3A and B, a very thin rudimentary coating 7 made of a material which contains titanium is applied to the basic compound 2. The latter can for example be a surgical suture thread, with which the healing process of the wound is accelerated by the coating 7. Due to the low thickness d of the rudimentary coating 7, this is not continuous on the surface of the basic compound 2, but is porous, as indicated in FIG. 3B, so that the open sections 8 are uncovered in order to form the coating-free zones on the implant. The body of the implant recipient can lead an absorption attack via these open sections. The basic compound 2 and the coating 7 of the implant 1″ consist in turn of the bioabsorbable materials or the PACVD-applied material which contains titanium already mentioned above.

With the thin rudimentary coating 7 which is not completely closed, the open sections 8 are generally irregular up to the given 5 nm due to the application of the coating with a thickness of a few atom layers, in contrast to which with the coating 4′, a regular arrangement of the interruptions 6 is more likely. The size of the interruptions 6 or open sections 8 is in each case essentially limited in terms of the minimum level by the technical feasibility, whereby for the masking, lithographic procedures are also possible, for example.

The use of the different types of coating 4, 4′ or 7 is primarily dependent on the respective area of use of the implant. When the implant is positioned between different contact surfaces, e.g. between soft tissue and a bone surface, a single-sided coating 4 with a directional orientation will generally be used. With implants which are located within a tissue type, such as suture material or so-called carrier systems, the masked coating 4′ or rudimentary coating 7 will tend to be used on the basic compound 2. Finally, the duration of the absorption of the implant 1, 1′, 1″ can be influenced via the ratio of the coated surface to the opening surface. In general, as well as titanium, as mentioned above, other metals which are compatible with the body such as tantalum, hafnium, zirconium, silver or niobium or biocompatible polymers, in particular hydroxyapatite can be used as a coating material.

What is claimed is:

1. An absorbable medical element suitable for insertion into the body, in particular an implant, comprising
a basic compound (2), which at least partially consists of a material which is absorbable in the body of the implant recipient, and
a coating (4, 4′, 7) on the basic compound (2) which coating (4, 4′, 7) contains a biocompatible material, is not absorbable and only covers the basic compound (2) partially in such a manner that the basic compound (2) comprises coating-free zones for an absorption attack by the body.

2. A medical element according to claim 1, wherein the partial coating is formed by a coating (4) which does not cover all sides (3, 5) of the medical element (1), and which is preferably on one side.

3. A medical element according to claim 1, wherein the coating (4′) has interruptions (6) in order to form the coating-free zones.

4. A medical element according to claim 3, wherein the interruptions (6) are formed by a corresponding masking of the basic compound (2) when the coating (4′) is applied.

5. A medical element according to claim 3, wherein the interruptions (6) are formed by a corresponding removal of the coating (4′) which has been applied.

6. A medical element according to claim 3, wherein the interruptions (6) comprise an open cross-section (q) in each case in the range of between a few μm and several mm.

7. A medical element according to claim 1, wherein the thickness (D) of the coating (4, 4′) lies in the range of between 5 and 700 nm, in particular between 10 and 100 nm.

8. A medical element according to claim 1, wherein the coating is designed as a thin, rudimentary coating (7), which comprises open sections (8) in order to form the coating-free zones.

9. A medical element according to claim 8, wherein the thickness (d) of the rudimentary coating (7) is approximately 5 nm.

10. A medical element according to claim 1, wherein the coating contains a metal which is compatible with the body, in particular titanium, tantalum, hafnium, zirconium, silver or niobium, or a biocompatible polymer, in particular hydroxylapatite.

11. A medical element according to claim 1, wherein the coating (4, 4′, 8) which contains titanium is applied using a PACVD procedure or a sol-gel procedure.

12. A medical element according to claim 1, wherein the basic compound (2) consists of polylactides, their copolymers, polyglycolides, their copolymers, polydioxanones, proteins or tricalcium phosphates.
